(19) United States
(12) Patent Application Publication
(10) Pub. No.: US 2013/0052726 A1
(43) Pub. Date: Feb. 28, 2013

(54) COMPOSTER

(71) Applicants: Kent Ashby, Logan, UT (US); Carl Russell Stanford, Clinton, UT (US); Clarissa M. Lindeman, Roy, UT (US); Neil Ben Watson, Layton, UT (US)

(72) Inventors: Kent Ashby, Logan, UT (US); Carl Russell Stanford, Clinton, UT (US); Clarissa M. Lindeman, Roy, UT (US); Neil Ben Watson, Layton, UT (US)

(73) Assignee: LIFETIME PRODUCTS, INC., Clearfield, UT (US)

(21) Appl. No.: 13/591,062

(22) Filed: Aug. 21, 2012

Related U.S. Application Data
(60) Provisional application No. 61/526,038, filed on Aug. 22, 2011.

(51) Int. Cl. C12M 1/10; C12M 1/00

(52) U.S. Cl. 435/290.3; 435/290.1

(57) ABSTRACT

In one example, a composter barrel includes a first panel having a first complementary structure, a second panel having a second complementary structure, and a connector element configured to connect the first and second panels together without the use of fasteners by engaging the first and second complementary structures.

### RELATED APPLICATIONS

This application claims priority to U.S. Provisional Patent Application Ser. 61/526,038, entitled COMPOSTER, filed Aug. 22, 2011, and incorporated herein in its entirety by this reference.

### BACKGROUND OF THE INVENTION

Embodiments of the present invention generally relate to composters of various types and configurations. In at least some embodiments, one or more portions of a composter component, such as a composter barrel for example, are configured to be connected together without the use of fasteners.

### BRIEF SUMMARY OF ASPECTS OF SOME EXAMPLE EMBODIMENTS

It should be noted that the embodiments disclosed herein do not constitute an exhaustive summary of all possible embodiments, nor does this brief summary constitute an exhaustive list of all aspects of any particular embodiment(s). Rather, this brief summary simply presents selected aspects of some example embodiments. It should further be noted that nothing herein should be construed as constituting an essential or indispensable element of any invention or embodiment. Rather, various aspects of the disclosed embodiments may be combined in a variety of ways so as to define yet further embodiments. Such further embodiments are considered as being within the scope of this disclosure. As well, none of the embodiments embraced within the scope of this disclosure should be construed as resolving, or being limited to the resolution of, any particular problem(s). Nor should such embodiments be construed to implement, or be limited to implementation of, any particular technical effect(s) or solution(s).

Example embodiments are concerned with composters. Example embodiments within the scope of this disclosure may include, among other things, one or more of the following, in any combination: a frame configured to accommodate one, two, or more, barrels; a handle that may enable a user to unlock a lid of a barrel with a single hand; a handle that may enable a user to unlock a lid of a barrel either way the handle is turned; a composter component that may have a partially, or completely, fastener-less construction; composter component having two or more elements that may be snap-fit together, either releasably or permanently; a composter component that may have two or more elements that may be held together, releasably or permanently, by an element other than a fastener; one or more blow-molded elements or components; means for holding two or more composter elements or components together without the use of fasteners; a composter barrel having two or more panels connected together without the use of fasteners; a composter barrel having six panels connected together without the use of fasteners; an adjustment mechanism; a handle mechanism; a barrel that includes one or more vents; an aeration bar; a non-cylindrical barrel; a processing indicator; and, a composter kit including a plurality of panels that, when connected together with one or more connector elements, form part of a barrel, the panels being connectable together without the use of fasteners.

### BRIEF DESCRIPTION OF THE DRAWINGS

The appended drawings contain figures of example embodiments to further illustrate and clarify the above and other aspects. It will be appreciated that these drawings depict only example embodiments of the invention and are not intended to be limiting in any way. Additionally, it will be appreciated that while the drawings may illustrate example sizes, scales, relationships and configurations of the invention, the drawings are not intended to be limiting in any way. Aspects of some example embodiments will be described and explained with additional specificity and detail through the use of the accompanying drawings in which:

FIG. 1 is a perspective view of an example composter;

FIG. 1a is a perspective view of an example composter with an open lid;

FIG. 2 is a front view of an example composter;

FIG. 3 is a back view of an example composter;

FIG. 4 is a side view of an example composter;

FIG. 5 is a top view of an example composter;

FIG. 6 is a partial exploded view of an example composter;

FIG. 7 is a detail view of example connector elements of a composter;

FIG. 7a is a detail exploded view of the elements shown in FIG. 7;

FIG. 8 is a partial perspective view of an example adjustment mechanism for a composter;

FIG. 9 is an exploded view of an example adjustment mechanism for a composter;

FIG. 10 is a partial perspective view of an example handle for a composter;

FIG. 11 is a partial perspective view of an example handle mechanism for a composter;

FIG. 12 is a perspective view of an example multi-barrel composter;

FIG. 13 is a front view of an example multi-barrel composter;

FIG. 14 is a back view of an example multi-barrel composter;

FIG. 15 is a side view of an example multi-barrel composter;

FIG. 16 is a top view of an example multi-barrel composter;

FIG. 17 is a partial top view of an interface area of a multi-barrel composter;

FIG. 18 is a partial perspective view of an example adjustment mechanism for a composter; and

FIG. 19 is an exploded view of the adjustment mechanism of FIG. 18.

### DETAILED DESCRIPTION OF SOME EXAMPLE EMBODIMENTS

Embodiments of the present invention are concerned with a composter, examples of which are disclosed in more detail herein. It will be appreciated that the composter may also have other suitable shapes, sizes, configurations and arrangements depending, for example, upon the intended use of the composter. It will also be appreciated that the composter does not have to include all of the various parts, components, features, aspects and characteristics disclosed herein, and it could include other parts, components, features, aspects and characteristics.

Additionally, to assist in the description of the composter, words such as top, bottom, front, rear, right and left are used to describe the accompanying figures. It will be appreciated, however, that one or more elements of embodiments of the present invention can be located in a variety of desired positions including various angles, sideways and even upside down.

### A. General Aspects of Some Example Embodiments

Any element of the composter that is constructed at least partly of blow-molded plastic, such as high-density polyethylene (HDPE) plastic for example, may have an interior that is partly, or completely, hollow. For example, elements of the composter may be integrally formed as part of a unitary, one piece structure during a blow-molding process. Such elements may also include, disposed in the interior, one or more depressions, sometimes referred to as “tack-offs.” In such embodiments, these tack-offs may be integrally formed as part of a unitary, one-piece structure during a blow-molding or other manufacturing process. One or more of the depressions may extend from a first surface, such as an exterior surface of the barrel for example, towards a second surface, such as an interior surface of the barrel for example. As well, one or more of the depressions may extend from a first surface, such as an interior surface of the barrel for example, towards a second surface, such as an exterior surface of the barrel for example.

The ends of one or more depressions may contact or engage the second surface, or the ends of one or more of the depressions may be spaced apart from the second surface by a distance. In some instances, one or more depressions on a first interior surface may or may not be substantially aligned with corresponding depressions on a second interior surface, and one or more depressions on the first interior surface may contact one or more corresponding depressions on the second interior surface or, alternatively, one or more depressions on the first interior surface may be spaced apart from corresponding depressions on the second interior surface. In still other instances, depressions that contact each other and depressions that are spaced apart from each other may or may not both be present in an element, such as a panel for example, of a composter. The depressions may be sized and configured to strengthen and/or reinforce the plastic element, or elements, of a composter.

While at least some embodiments of the composter may include one or more elements constructed of plastic using a blow-molding process, the use of blow-molded plastic elements in the composter is not required. For example, processes such as roto-molding and/or processes sometimes referred to as “twin sheet” processes may be used to construct one or more plastic elements of a composter. At least some embodiments of the composter may include a mixture of one or more of blow-molded, vacuum molded, roto-molded, drape molded, and/or twin sheet formed plastic elements. Yet other embodiments of the composter may include only a few, or no, plastic elements.

As well, the foregoing, and other, composter components disclosed herein may be constructed with a variety of components and materials including, but not limited to, plastic (including blow-molded plastic structures and elements), including polycarbonates, composites, metals, and combinations of any of the foregoing. One example plastic is an HDPE plastic that may be bisphenol A (BPA)-free, or substantially BPA-free. In some instances, the plastic may be a dark color that may or may not be black. Suitable metals may include steel, aluminum, and aluminum alloys, although the skilled person will understand that a variety of other metals may be employed as well and the scope of the invention is not limited to the foregoing examples. Where metal is employed in the construction of a composter component, the metal elements may take one or more forms including, but not limited to, pipe, square tube, rectangular tube, round tube, pipe, angles, flatbar, I-shapes, T-shapes, L-shapes, and combinations and portions of any of the foregoing.

Depending upon the material(s) employed in the construction of the composter, a variety of methods and components may be used to connect, releasably or permanently, various elements of the composter. For example, the various elements of a composter or composter component within the scope of this disclosure may be attached to each other by any one or more of allied processes such as welding or brazing, and/or mechanically by way of fasteners such as bolts, screws, pins, and rivets, for example. In some instances, various components of the composter may be configured to be connected to each other, permanently or removably, with few, or no, fasteners.

Finally, some, none, or all of the portions of a composter may be coated, or otherwise covered with paint, rubber, plastic, galvanizing, powder coating or other materials, or any combination of the foregoing. Surface treatments and textures may also be applied to portions of the composter. At least some of such materials may serve to help prevent, or reduce, rust and corrosion.

### B. Some Example Composters—Single Barrel

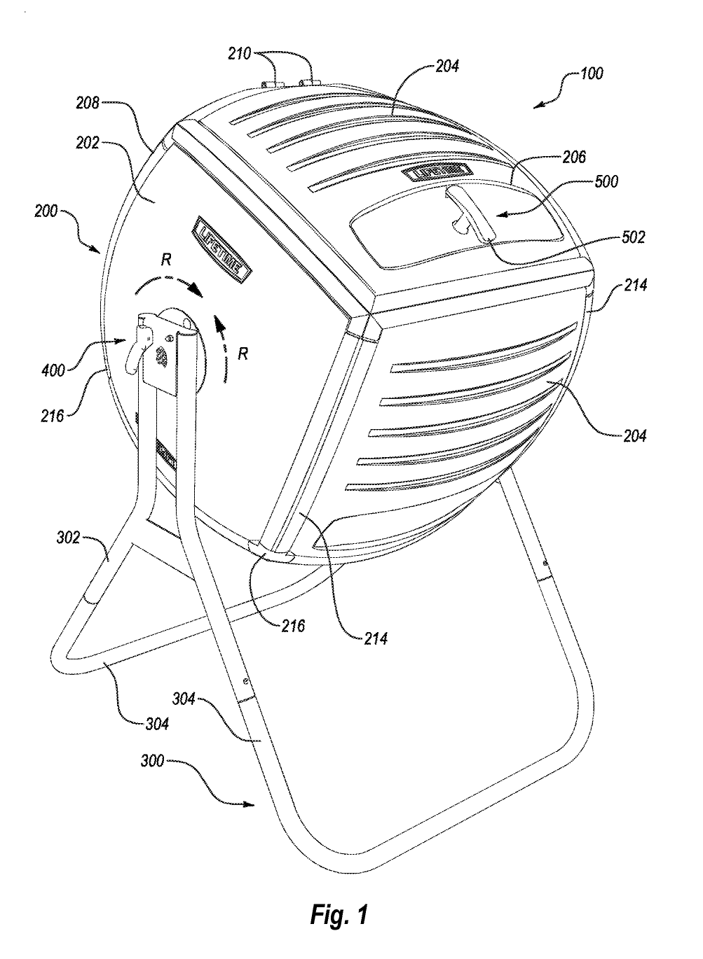

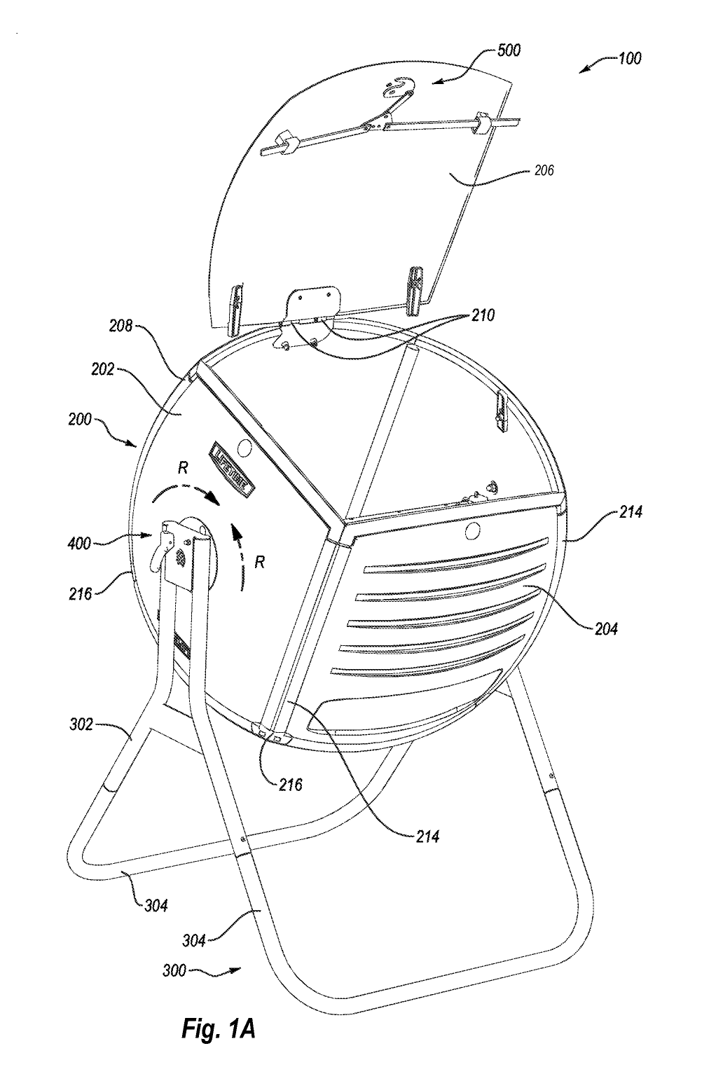

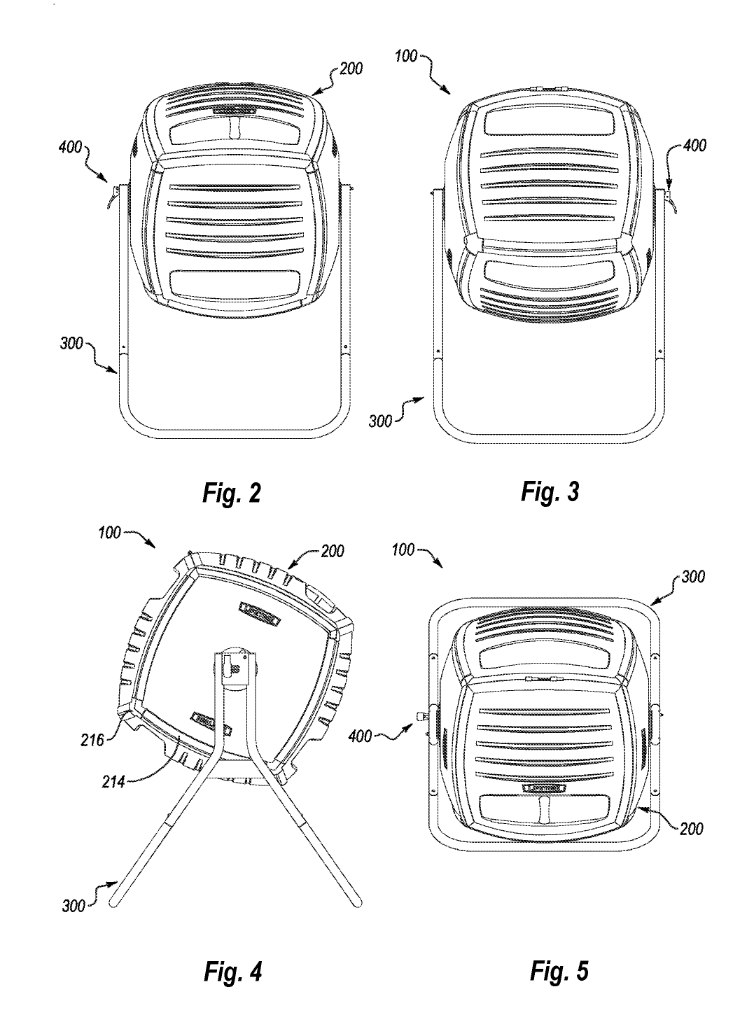

With particular reference initially to FIGS. 1-5, an example of a composter 100 is disclosed. In general, the composter 100 may include one or more barrels 200 supported by a frame 300. In at least some embodiments, the barrel 200 is rotatably supported by the frame 300, and a suitable shaft, bearings, and other components (not shown) may be provided for that purpose. An adjustment mechanism 400 may be provided that, among other things, permits a user to releasably secure the barrel 200 in a desired position. The use of relatively lightweight components, such as tubular metal components and blow-molded plastic components for example, may enable some embodiments of the composter 100 to be relatively light weight and portable. Finally, a handle mechanism 500 may be provided that operates in conjunction with a lid so as to enable a user to add materials to, and remove materials from, the composter 100, while also securing the lid shut.

### C. Aspects of Some Example Barrels

As indicated in the Figures, embodiments of the composter 100 may include one or more barrels 200, and the following discussion applies both to single barrel and multiple barrel configurations. The barrels used in the single barrel configuration may or may not be similar, or identical, to one or more barrels employed in a multiple barrel configuration.

While not specifically illustrated, some embodiments of the barrels 200 may include a plurality of storage compartments. The multiple compartments may allow composting of different materials and composting at different times. For example, one compartment may be used to create fresh compost that may be formed in two to three weeks, while another compartment may be used to create cured compost that may be formed in two to three months. The multiple compartments may also allow different materials to be composted and the materials may be composted at different rates. For instance, a first compartment may be filled with desired amounts and types of materials, and these materials may be allowed to compost for a period of time. A second compartment may be filled at the same or different time, and it may be filled with the same or different amounts and types of materials, and these materials may be allowed to compost for a length of time. Thus, the multiple compartments may allow different materials to be composted, different types and amounts of compost to be created, different degrees of composting to be completed, etc. This may also allow the composting process to be completed sooner or at different times.

As well, some embodiments of the composter 100 may include one or more barrels 200 with one or more partitions (not shown) to divide the barrel 200 into storage compartments of equal or different sizes. The partitions may be movable to allow the size of the compartments to be adjusted. In addition, the partitions may be used to completely separate the compartments, or the partitions may allow some communication or interaction between the compartments. The partitions may be constructed from metal (such as steel or aluminum), plastic (such as vinyl or nylon), fabric (such as cloth or polyester), or other suitable natural or synthetic materials. The partitions may be solid structures or they may include openings, holes, apertures or the like to allow communication between the compartments. Among other things, such openings may permit fluids, solids and/or gases to pass from one compartment to another.

The barrel 200 may be implemented in a variety of different shapes, and barrels of different shapes may be combined together in a single composter. In the example disclosed in FIGS. 1-5, the barrel 200 includes four sides, one of which may serve as a lid, that collectively define a substantially non-cylindrical configuration, when the barrel 200 is viewed from the end (see FIG. 4). As a consequence of this configuration, the interior of the barrel 200 may have a non-cylindrical configuration. The non-cylindrical shape may help to ensure better mixing of the compost materials as the barrel 200 is rotated. As indicated in the particular example of FIG. 4, the barrels 200 may be cube-shaped, or generally cube-shaped. A generally cube-shaped configuration may or may not have one or more sides that curve outwardly.

Of course, the barrels 200 may take other substantially non-cylindrical forms, such as elliptical for example, and the scope of the invention is not limited to any particular non-cylindrical barrel configuration. While the example barrel 200 has four sides, other embodiments of the barrel may have more, or fewer, sides that cooperate to at least partly define a substantially non-cylindrical configuration. In yet other embodiments, one or more barrels 200 may be substantially cylindrical in shape.

As with other aspects of example embodiments of the barrel 200, the color of the barrel 200 may vary in different embodiments. In at least some embodiments, the barrel 200 is dark, or black. Dark colors may help the composter 100 to absorb heat so as to aid in the composting process. In some instances, the barrel 200 is substantially the same color throughout while, in other embodiments, different portions of the barrel 200 may have different colors.

The barrel 200 may be of any suitable volume. In some embodiments, the barrel 200 may have a volume of about 50 gallons, although larger or smaller volumes may be employed. For example, a single barrel composter may have a barrel with a volume of about 50 gallons. Where multiple barrels 200 are employed in a single composter 100, the barrels 200 may or may not have the same volume as each other, and one or more of those barrels may have a volume of about 50 gallons, although larger or smaller volumes may be employed.

Finally, and as disclosed in more detail herein, the barrel 200 may be vented so as to facilitate airflow through the interior of the barrel 200 that can be helpful to the composting process. One or more vents may be located at any suitable location(s) in the barrel 200.

### D. Aspects of Some Example Panels

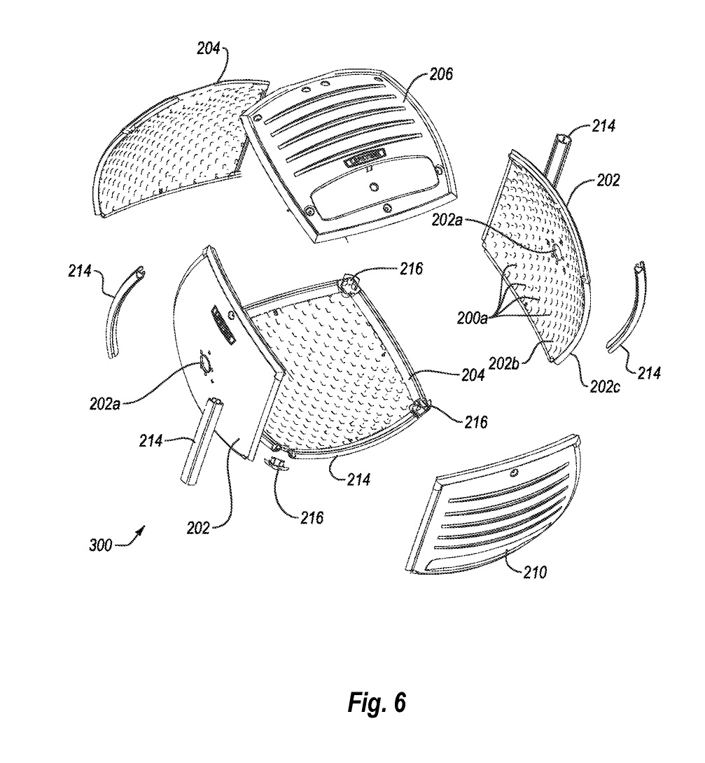

The following discussion applies both to single barrel and multiple barrel configurations. With more particular reference to the barrel 200, and directing attention now also to FIG. 6, example embodiments of the barrel 200 may comprise a plurality of panels that can be attached to each other and/or to other components with few, or no, fasteners such as screws and bolts. In one embodiment, the barrel 200 includes two end panels and four side panels, all of which are connected together without the use of fasteners. The fastener-less configuration may be advantageous in some instances insofar as it may obviate the need for tools to assemble the barrel, may permit the barrel to be easily disassembled, and/or may permit ready replacement of a damaged panel.

In the particular example of FIG. 6, the barrel 200 includes first and second end panels 202 which may, in some embodiments, have substantially the same configuration as each other. A portion of the adjustment mechanism 400 may be attached to each end panel 202, and the end panels 202 may each define an opening 202a, which may be reinforced with metal and/or other materials, through which a corresponding end of shaft (not shown) passes. In addition to the end panels 202, the barrel 200 may also include one or more side panels 204, one or more of which may serve as a lid, such as lid 206. One or more end panels 202 and/or side panels 204 may be substantially planar in shape, curved, or have any other desired configuration.

The example barrel 200 of FIGS. 1-7 includes four side panels 204 and two end panels 202, although other embodiments may include more or fewer side panels 204 and/or more or fewer end panels 202. One or more of the side panels 204 and/or end panels 202 may be constructed of blow-molded plastic and may include one or more tack-offs 206 (see, e.g., FIG. 11 for example), although blow-molded plastic is not required to be used for any of the side panels 204 or end panels 202. As noted elsewhere herein, one or more of the side panels 204 and/or end panels 202 may be attached to another side panel 204 with only a few, or no, fasteners.

With continued reference to FIGS. 1-5, and as noted above, the barrel 200 may include a lid 206 attached to a body 208 of the barrel by one or more hinges 210 or other suitable mechanism(s). The lid 206 may be securely connected to create a water-tight seal, which may help prevent rain from entering the composter 100 and it may help maintain the desired moisture within the composter 100. As discussed in more detail elsewhere herein, the lid 206 may have a construction that is similar to that of other portions of the barrel 200, such as the side panels 204 for example.

As best shown in FIG. 6, one or more of the end panels 202 and/or side panels 204, including lid 206, may include a plurality of elements 200a that may extend toward, or away from, the interior of the barrel 200. The elements 200a may be integrally formed with the panel as part of a blow-molding, or other, manufacturing process. In the particular example of FIG. 6, at least some of the elements 200a take the form of depressions, inasmuch as one or more of the elements 200a may extend away from an inner surface 202b of a panel 202 toward an outer surface 202c of a panel 202. In some instances, one or more of the elements 200a may extend outwardly from the inner surface 202b toward the interior of the barrel 200.

In some embodiments, elements 200a extending away from an inner surface 202b of a panel 202 toward an outer surface 202c of a panel 202 may be combined with elements 200a extending outwardly from the inner surface 202b toward the interior of the barrel 200. This combination may be implemented in one or more of the panels. In some embodiments, a panel may have only elements 200a extending away from an inner surface 202b of a panel 202 toward an outer surface 202c of a panel 202, or may have only elements 200a extending outwardly from the inner surface 202b toward the interior of the barrel 200. One or more panels with only elements 200a extending away from an inner surface 202b of a panel 202 toward an outer surface 202c of a panel 202 may be combined together in a single composter with one or more panels having only elements 200a extending outwardly from the inner surface 202b toward the interior of the barrel 200.

The elements 200a can take any desired form. As well, the size, number, location, geometry, and orientation of the elements 200a may be implemented as desired. In at least some embodiments, one or more of the end panels 202 and/or side panels 204 may include a plurality of elements 200a that each have substantially the same geometry and which are substantially evenly, or randomly, distributed over some or all of an interior surface of the panel. One, some, or all of the elements 200a may be implemented as tack-offs.

### E. Aspects of Some Example Connector Elements

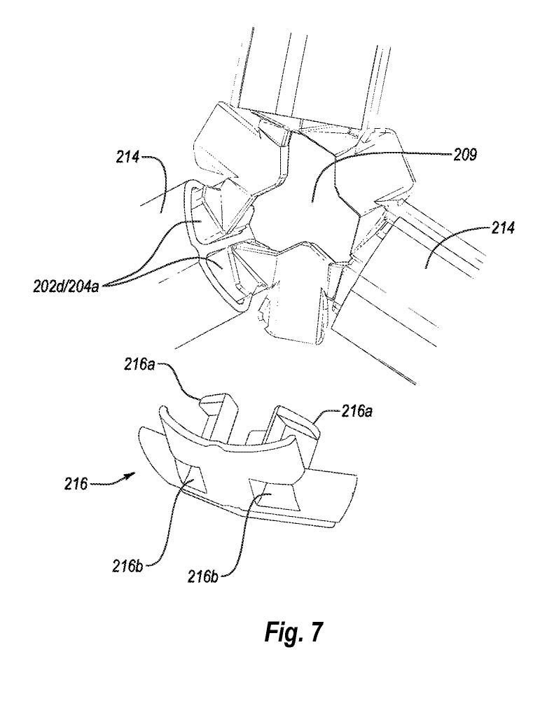

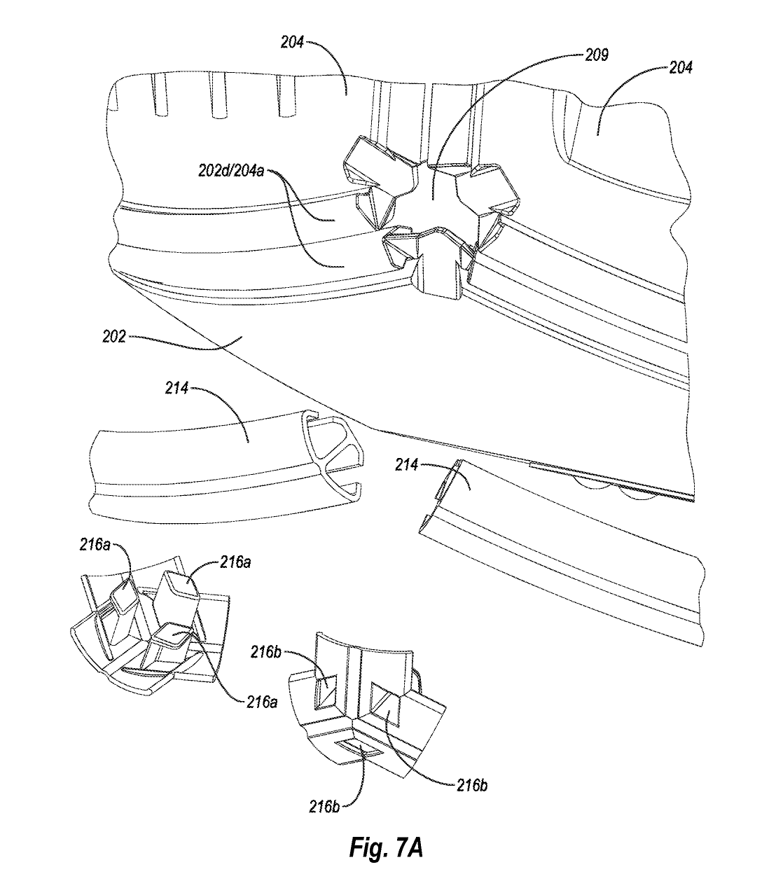

The following discussion applies both to single barrel and multiple barrel composter configurations. With continued attention to FIG. 6, and directing attention now to FIGS. 7, 7a and 11 as well, details are provided concerning some example connector elements 214 and 216 that may be employed to connect panels of the barrel 200 without the use of fasteners. The connector elements 214 and/or 216 may be configured to permanently, or releasably, connect one barrel component to another barrel component, where such barrel components include, for example, end panels 202 and side panels 204. Similar to some embodiments of the side panels 204 and end panels 202, one or more connector elements 214 and/or 216 may be constructed of blow-molded plastic, although other materials, such as metal or extruded plastic for example, may be employed in the construction of the connector elements 214 and/or 216. In the example of FIG. 6, the connector elements 214 comprise side connector elements, and the connector elements 216 comprise corner connector elements.

As best shown in FIG. 11, adjacent end panels 202 and side panels 204 may each include respective engagement portions 202d and 204a. These engagement portions 202d and 204a may be configured in any manner that permits their engagement with a connector element 214. In general then, and as discussed with reference to specific examples below, the end panels 202 and side panels 204, and connector elements 214, all have complementary structures that enable the end panels 202 and side panels 204 to be connected to other end panels 202 and side panels 204 using connector elements 214, and with few, or no, fasteners.

In the example of FIGS. 7 and 11, the engagement portions 202d and 204a may include a relatively wide portion connected to the corresponding end panel 202 or side panel 204, as applicable, by a relatively narrow neck portion although, as noted above, any suitable configuration of an engagement portion can be employed. These engagement portions, and the channels 214A discussed below, are examples of complementary structures, and are also example structural implementations of a means for connecting panels without the use of fasteners. However, any other structure(s) capable of implementing comparable functionality are considered to be within the scope of this disclosure.

In any case, thus configured, one or more of the engagement portions 202d/204a may be configured such that when brought in close proximity to each other, the engagement portions 202d/204a can be received, slidingly for example, in corresponding channels 214A defined by the connector element(s) 214. Alternatively, one or more of the engagement portions 202d and/or 204a may be snap-fit into the channels 214A of a connector element 214. As a consequence of this construction, an end panel 202 or side panel 204 can be connected to another end panel 202 and/or side panel 204 without the use of fasteners. In at least some embodiments, this construction also permits an end panel 202 and/or side panel 204 to be readily detached from another end panel 202 and/or side panel 204. In at least some embodiments, all of the end panels 202 and side panels 204 are connected together using connector elements 214, although in other embodiments, fewer than all of the end panels 202 and side panels 204 are connected together using connector elements 214. As well, any number of end panels 202 and/or side panels 204 can be connected to one or more other end panels 202 and/or side panels 204.

While, in some embodiments, one or more end panels 202 and/or side panels 204 may be removably connected to another end panel 202 and/or side panel 204, yet other embodiments provide for the use of adhesives or other materials, in conjunction with one or more connector elements 214, to permanently connect one or more end panels 202 and/or one or more side panels 204 to one or more other end panels 202 and/or one or more side panels 204.

With particular reference now to FIG. 7, embodiments of the barrel 200 may include one or more connector elements 216. As indicated in FIG. 1, for example, connector elements 216 may be employed at the intersection of one or more end panels 202 and one or more side panels 204. The connector element(s) 216 may or may not be employed in conjunction with connector elements 214. As in the case of connector elements 214, connector elements 216 may facilitate the permanent or releasable connection of one or more end panels 202 and/or one or more side panels 204 with one or more other end panels 202 and/or side panels 204, without necessitating the use of fasteners. For example, connector elements 216 may serve to releasably, or permanently, connect one or more connector elements 214 together and/or one or more panels 202/204 together. In one particular example, a connector element 216 may be a corner connector element that releasably, or permanently, connects three connector elements 214 together, where the three connector elements 214 may each take the form of a side connector element.

In the example of FIGS. 1 and 1a, a total of four connector elements 216, one at each corner of the barrel 200, are provided and, in this example, connector elements 216 are not attached to the lid 206 in order to enable the lid 206 to move freely relative to the rest of the barrel 200.

In the example of FIG. 7, the connector elements 216, end panels 202 and side panels 204 may each have complementary structures that enable the connector elements 216 to engage, permanently or releasably, one or more end panels 202 and/or side panels 204 and retain those end panels 202 and/or side panels 204 in a desired configuration. These complementary structures, which may be implemented in any suitable form, are also example structural implementations of a means for connecting panels without the use of fasteners. However, any other structure(s) capable of implementing comparable functionality are considered to be within the scope of this disclosure.

In one particular embodiment, the connector elements 216 may have one or more protrusions 216A configured to be received in corresponding recesses 209 defined, individually or collectively, by one or more end panels 202 and/or one or more side panels 204. The protrusions may be permanently, or releasably, received in the corresponding recesses. The aforementioned configuration may be reversed such that the connector elements 216 include the recesses, while one or more end panels 202 and/or one or more side panels 204 include the protrusions. In yet other embodiments, one or more of the connector elements 216, end panel(s) 202 and side panel(s) 204 may include both recesses and protrusions.

As noted elsewhere herein, at least some embodiments of the composter include one or more vents to facilitate air flow through the interior of the composter. Such vents may be located anywhere in the composter. Accordingly, some implementations of the connector element 216 may include one or more vents 216b that may enable air to flow through the interior of the composter 100 by way of the connector element 216. Additionally, or alternatively, other elements of the composter 100, such as one or more end panels 202 and/or side panels 204 for example, may include one or more vents.

In at least some embodiments, the connector element 216 is configured to be snap-fit together with one or more end panels 202 and/or one or more side panels 204, although other fit types may be employed. As in the case of the connector elements 214, the connector elements 216 may be used in conjunction with adhesives or other materials such that connector elements 216 are permanently retained in place.

As evidenced by the discussion of connector elements 214 and 216, at least some embodiments of the barrel 200 may be substantially, or completely, assembled without the use of fasteners. As well, the use of connector elements such as connector elements 214 and/or 216 may enable the composter to be easily assembled. For example, the composter may be disposed in a first configuration for shipping, storage and/or transport and a second configuration for use. Desirably, the composter may be at least partially unassembled in the shipping configuration. In addition, two or more components of the composter may at least partially nest together in the shipping configuration, which may help reduce the size of the packaging necessary to ship the composter and that may help reduce shipping costs. The composter may also be quickly and easily disassembled for storage, if desired.

### F. Aspects of An Example Frame and Associated Elements

With continued reference to FIG. 1, and particularly the frame 300, at least some embodiments of the frame 300 may include a barrel support portion 302 connected to one or more legs 304, although other configurations may be employed. The barrel support portion 302 and/or the legs 304 may comprise flat metal elements and/or tubular metal elements, such as aluminum or steel for example, of any cross-sectional shape including, for example, round, square, or oval. The legs 304 may be permanently, or removably, attached to the barrel support portion 302.

As noted above, the barrel 200 may be rotatably supported by the frame 300. Particularly, and with reference now to FIG. 9, a shaft 201 rotatably supported by the frame 300 at first and second ends of the shaft, passes through the barrel 200 and thus defines an axis of rotation for the barrel 200. Bearings (not shown) may or may not be employed for enabling rotatable support of the shaft 201. In at least some embodiments, the shaft 201 is metal, and may be tubular or solid. The shaft 201 may be fixed to the frame 300 such that the barrel 200 can rotate about the fixed shaft 201. Alternatively, the shaft 201 may be fixed to the barrel 200 and rotatably supported by the frame 300, such as by way of bracket 301, such that the barrel 200 and shaft 201 rotate in unison with each other.

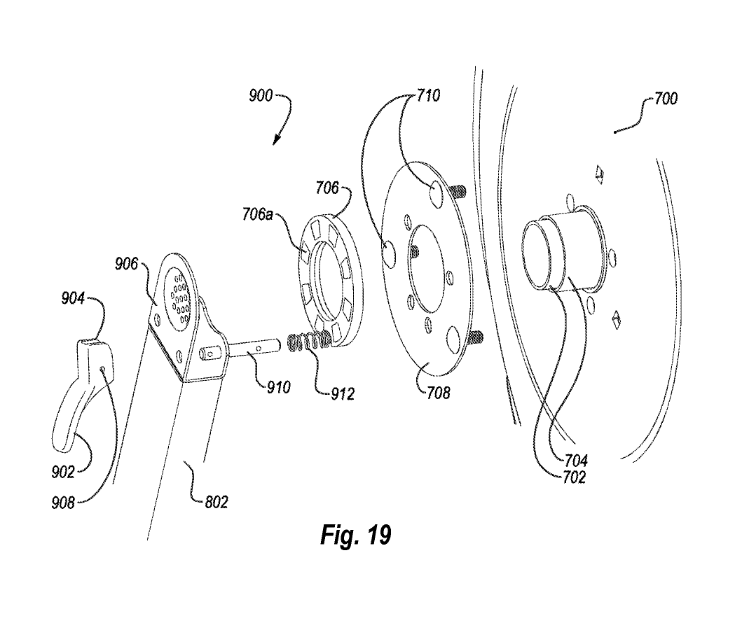

With brief reference to the example of FIG. 19, the shaft 702 may pass through an aeration bar 704 that is at least partially disposed in the barrel 700. The aeration bar 704 may be hollow, and include one or more openings (not shown in FIG. 19) to facilitate gas flow within the barrel 700 and/or allow outside air to be introduced into, and pass through, the barrel 700. As well, the aeration bar 704 may serve to agitate material in the barrel 700 as the barrel 700 is rotated. Some embodiments of the composter 100 may include a plurality of such aeration bars, or other elements of comparable functionality. In some embodiments, such as the example of FIG. 9, the aeration bar 201b may also serve as a shaft that is fixed either to the barrel 200 or to the frame 300, thereby combining the functionality of a shaft and aeration bar into a single element. The aeration bar 201b may include one or more openings 201c to facilitate gas flow within the barrel 200 and/or allow outside air to be introduced into, and pass through, the barrel 200.

### G. Aspects of Some Example Adjustment Mechanisms

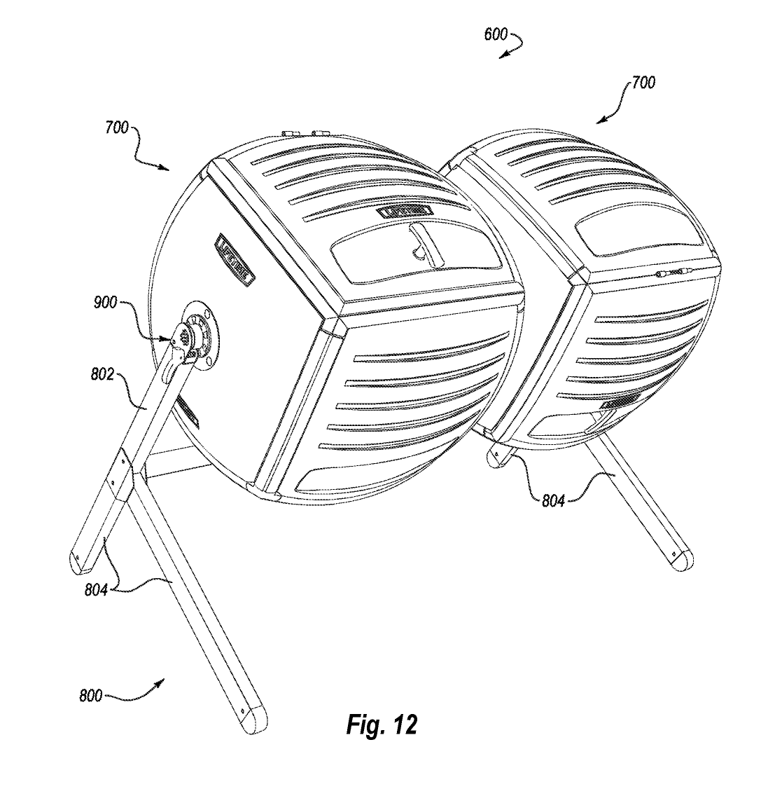

As disclosed elsewhere herein, embodiments of the composter 100 may include an adjustment mechanism, such as adjustment mechanism 400 or adjustment mechanism 900 (see FIG. 12). In general, the adjustment mechanism may enable a user to releasably secure the barrel in a desired position. When not secured in position by the adjustment mechanism, the barrel is free to rotate. As suggested, for example, by the arrows denoted with ‘R’ in FIGS. 1 and 1a, the rotation of the barrel may occur in either direction.

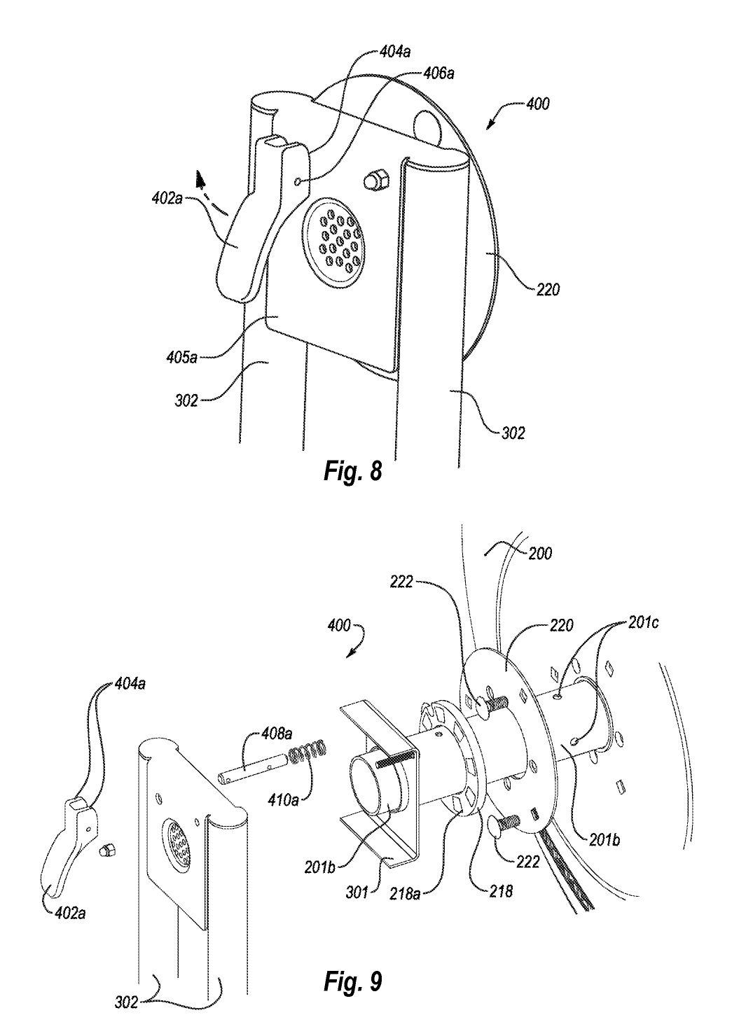

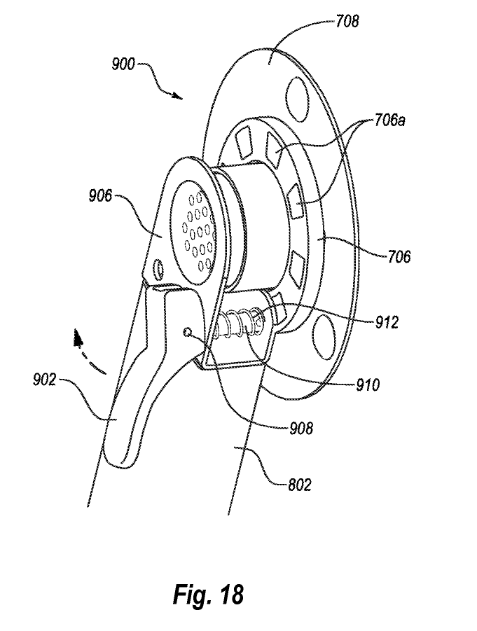

With more particular reference now to FIGS. 8 and 9, details are provided concerning aspects of examples of the adjustment mechanism 400 (adjustment mechanism 900 is discussed below in connection with FIGS. 18 and 19). In general, the adjustment mechanism 400 disclosed in FIGS. 8 and 9 may be employed in conjunction with a single barrel composter, and the adjustment mechanism 900 may be employed in conjunction with a multi barrel composter, although neither adjustment mechanism is required to be employed with any particular composter configuration. The components of the adjustment mechanisms disclosed herein may be constructed of any suitable material(s), including metals such as aluminum and steel.

With regard first to FIGS. 8 and 9, one or more elements of the adjustment mechanism 400, which may be attached to the barrel support portion 302 of the frame 300, may engage an adjustment plate 218 of the barrel 200. The adjustment plate 218 may be located adjacent a support plate 220 that is bolted to the barrel 200 with bolts 222, or otherwise attached to the barrel 200, and supports the adjustment plate 218. The adjustment plate 218 may be configured for rotation in unison with the barrel 200 and include a plurality of depressions 218A, which may be implemented as holes, that each correspond to a particular rotational position of the barrel 200 to which the adjustment plate 218 is attached.

A handle 402A of the adjustment mechanism 400 includes a cam portion 404A which bears against plate 405A such that rotation of the handle 402A about pin 406A causes a corresponding linear motion, such as extension or retraction, of an adjustment pin 408A to which the pin 406A is connected. More specifically, by rotating the handle 402 upward, in the direction indicated by the arrow in FIG. 8, the user can retract the adjustment pin 408A from a depression 218a of the plate 218, thereby freeing the plate 218 and, thus, the barrel 200, to rotate to another position.

Rotation of the handle 402 downward to the position indicated in FIG. 8 moves the adjustment pin 408 to a retracted position wherein a portion of the adjustment pin 408 can reside in one of the depressions 218A, thus retaining the barrel 200 in a desired position by preventing rotational motion of the barrel 200. As explained in connection with the embodiment of FIG. 12, the adjustment mechanism 400 may include a biasing element 410A, such as a spring for example, to bias the adjustment pin 408 in a desired direction, such as toward, or away from, the adjustment plate 218. In this regard, the adjustment pin 408 may include a plate or other element arranged to enable the biasing element 410 to exert a force on the adjustment pin 408 in a direction at least approximately parallel to a longitudinal axis of the adjustment pin 408. In one particular embodiment, the biasing element 410 biases the adjustment pin 408 toward the adjustment plate 218. More particularly, the biasing element 410 may bias the adjustment pin 408 into a depression defined by the adjustment plate 218.

### G. Aspects of An Example Handle Mechanism

The following discussion applies both to single barrel and multiple barrel configurations. As noted elsewhere herein, a handle mechanism 500 may be provided that is connected with the lid 202 and, as discussed in further detail elsewhere herein, may enable a user to unlock and lock the lid 206 by turning a handle 502 in either direction.

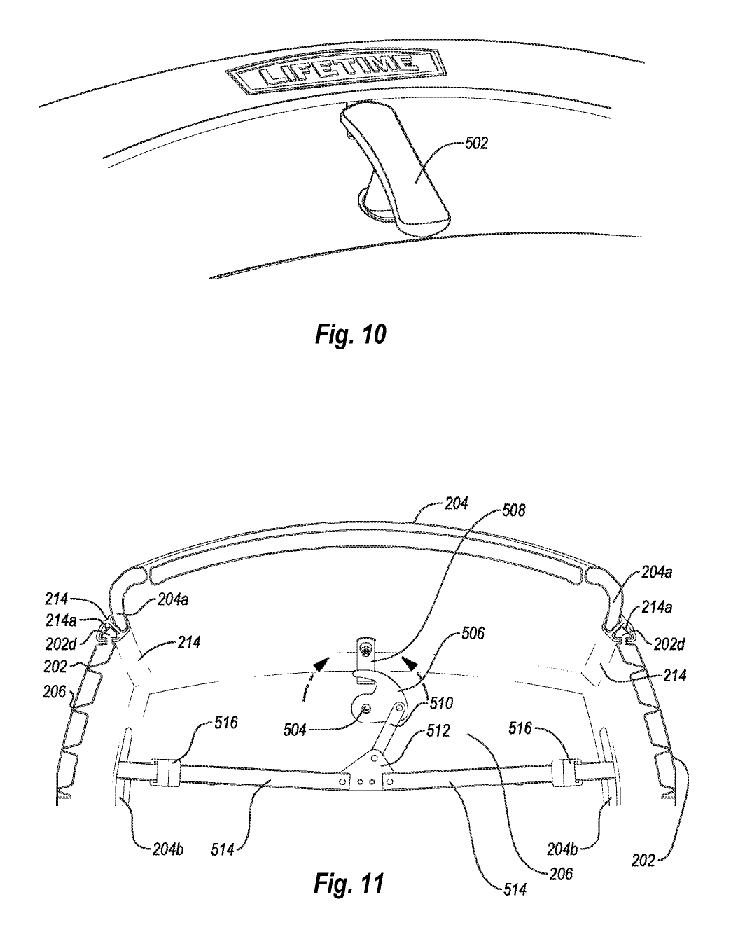

With particular attention now to FIGS. 10 and 11, details are provided concerning an example of a handle mechanism 500, whose various components may comprise materials such as plastic, metals such as aluminum or steel, and/or other suitable materials. As noted elsewhere, the handle mechanism 500 includes a handle 502 attached to a pin 504 that extends through the lid 206 and is fixed to a latch 506 that is configured to selectively engage a locking tab 508 attached to side panel 204. A latch arm 510 rotatably connects the latch 506 to a pivot plate 512. A pair of locking arms 514 are rotatably connected to the pivot plate 512 and are configured for reciprocal motion into, and out of, lock slots 204b defined by the body 208 of the composter 200. A pair of support brackets 516 are attached to the interior of the body 208 and support the locking arms 514.

In operation, rotation of the latch 506 may be effected by rotation of the handle 502. A clockwise (with reference to FIG. 9) rotation of the latch 506 disengages the latch 506 from the locking tab 508, and also causes a downward motion of the latch arm 510 and, thus, the pivot plate 512. The downward motion of the pivot plate 512 causes retraction of the locking arms 514 from their respective lock slots 204B. Thus, the clockwise rotation of the handle 502 unlocks the lid 202 from the body 208. A counterclockwise rotation of the handle 502 engages the latch 506 with the locking tab 508, and also causes an upward movement of the pivot plate 512 such that the locking arms 514 are caused to rotate into a position where they extend into their respect lock slots 204B, thus locking the lid 206 to the body 208.

### H. Some Example Composters—Multiple Barrel

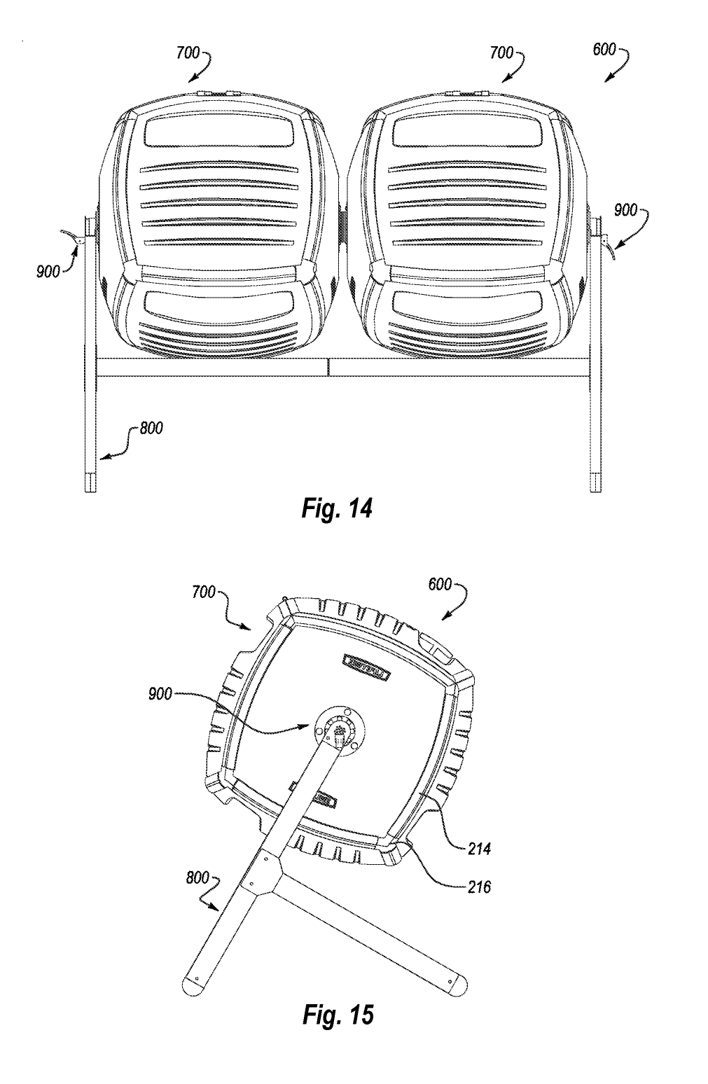

As noted elsewhere herein, embodiments of a composter may include a single barrel, or multiple barrels. An example of a multi-barrel configuration is disclosed in FIGS. 12-19. While that configuration includes two barrels, yet other embodiments may include more than two barrels. Moreover, while FIGS. 12-19 disclose a composter 600 that includes multiple barrels in a side-by-side configuration, the scope of this disclosure is not limited to such configurations. For example, some embodiments of the composter include one or more barrels and a frame that is configured to support the barrels in a ‘Ferris wheel’ type of configuration where each barrel may be rotatable about its own axis, and also about another axis defined by the frame. Such a configuration may be useful where there is limited ground space to locate the composter.

In the multi-barrel configuration, the composter may include barrels that are all substantially the same size and/or configuration, or barrels of different sizes may be mixed within a given composter implementation. As is the case of the single barrel composters disclosed herein, the sizes of the barrels in a multi-barrel configuration may be selected as desired. In one example embodiment, a multi-barrel composter includes two barrels, each having a capacity of approximately fifty gallons Likewise, an example embodiment of a single barrel composter includes a single barrel having a capacity of approximately fifty gallons. These sizes and configurations are presented solely by way of example however, and the scope of this disclosure is not limited to composter barrels of any particular capacity or range of capacities. By way of illustration, composters within the scope of this disclosure may include one or more barrels having a capacity less than, or greater than, fifty gallons. Moreover, the barrels of the single and multi-barrel composters disclosed herein may be interchangeable such that a user can readily change the capacity of the composter by simply replacing a barrel of one size with a barrel of another size.

With particular reference now to FIGS. 12-19, details are provided concerning aspects of an example embodiment of a multi-barrel composter 600. The multi-barrel composter 600 includes a plurality of barrels 700 supported by a frame 800. It should be noted that one or more of the barrels 700 may be similar, or identical, to the barrels 200.

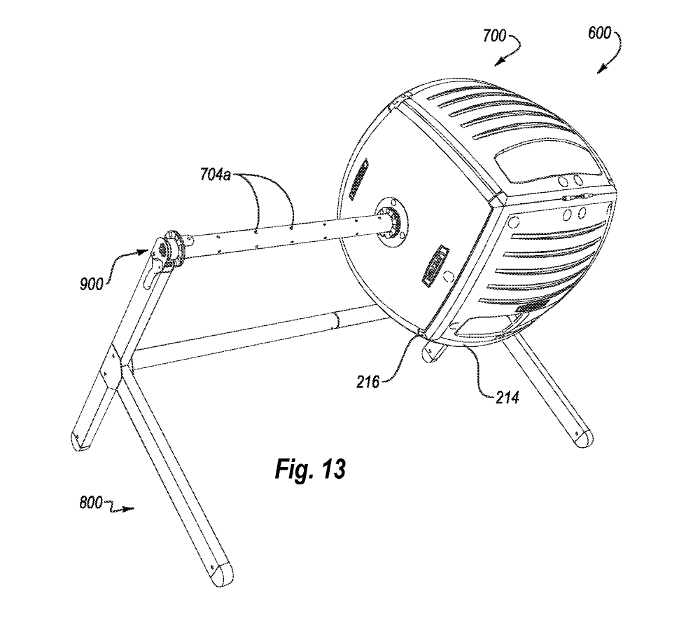

As in the case of the single barrel composters disclosed herein, the barrels 700 and/or an associated shaft 702 (see FIG. 19) passing through the barrels 700 may be rotatably supported such that the barrels 700 can rotate relative to the shaft, or the barrels 700 can be fixed to the shaft such that the barrels 700 and shaft 702 rotate in unison relative to the frame 800. The shaft 702 may or may not pass through an aeration bar 704 that is at least partially disposed in the barrel 700. The aeration bar 704 may be hollow, and include one or more openings 704a (see FIG. 13) to facilitate gas flow within the barrel 700 and/or allow outside air to be introduced into, and pass through, the barrel 700.

The barrels 700 may be constructed and configured as disclosed elsewhere herein and, as such, may include one or more of a lid, a body, a hinge, and a handle mechanism, end panels, side panels, tack-offs, connector elements. As well, the barrels 700 and/or frame 800 may be configured so as to enable the barrels 700 to rotate in unison with each other and/or independently of each other.

With particular reference now to FIGS. 12-16, the frame 800 may include one or more barrel support portions 802 and legs 804, and the frame 800 may be constructed at least partly of hollow metal elements such as round tube, square tube and/or rectangular tube. Examples of metals that may be suitable for the construction of the frame 800 include aluminum and steel. As is the case with the frame 300, elements of the frame 800 may be attached together in any suitable matter, such as by welding, brazing and/or with fasteners such as screws and bolts. Correspondingly, one or more elements of the frame 800 may be permanently, or removably, attached to each other. The frame 800 may be constructed so as to hold the barrels 700 in a desired position and/or orientation relative to each other and/or reference surfaces such as the ground.

Turning now to FIGS. 13, 18 and 19, each barrel 700 may have a dedicated adjustment mechanism 900, discussed in more detail below, such that each barrel 700 can be rotated independently of the other. The adjustment mechanism 900 may be configured, and operate, in a manner similar, or identical, to the adjustment mechanism 400.

With particular reference to FIGS. 18 and 19, details are provided concerning the example adjustment mechanism 900, which may operate with similar effect to adjustment mechanism 400 described above. As in the case of adjustment mechanism 400, one or more elements of the adjustment mechanism 900, which may be attached to the barrel support portion 802 of the frame 800, may engage an adjustment plate 706 of the barrel 700 (removed for clarity). The adjustment plate 706 may be configured for rotation in unison with the barrel 700 and include a plurality of depressions 706a, which may be implemented as holes, that each correspond to a particular rotational position of the barrel 700 to which the plate adjustment plate 706 is attached. The adjustment plate 706 may be located adjacent a support plate 708 that is bolted to the barrel 700 with bolts 710, or otherwise attached to the barrel 700, and supports the adjustment plate 706.

A handle 902 of the adjustment mechanism 900 includes a cam portion 904 which bears against plate 906 such that rotation of the handle 902 about pin 908 causes a corresponding linear motion, such as extension or retraction, of an adjustment pin 910 to which the pin 908 is connected. More specifically, by rotating the handle 902 upward, in the direction indicated by the arrow in FIG. 18, the user can retract the adjustment pin 910 from a depression 706a defined in the plate 706, thereby freeing the plate 706 and, thus, the barrel 700, to rotate to another position. Rotation of the handle 902 downward to the position indicated in FIG. 12 moves the adjustment pin 910 to a retracted position wherein a portion of the adjustment pin 910 can reside in one of the depressions 706a, thus retaining the barrel 700 in a desired position by preventing rotational motion of the barrel 200. The adjustment mechanism 900 may include a biasing element 912, such as a spring for example, to bias the adjustment pin 910 in a desired direction, such as toward, or away from, the adjustment plate 706. In this regard, the adjustment pin 910 may include a plate or other element (not shown) arranged to enable the biasing element 912 to exert a force on the adjustment pin 910 in a direction at least approximately parallel to a longitudinal axis of the adjustment pin 910. In one particular embodiment, the biasing element 912 biases the adjustment pin 910 toward the adjustment plate 706. More particularly, the biasing element 912 may bias the adjustment pin 910 into a depression defined by the adjustment plate 706.

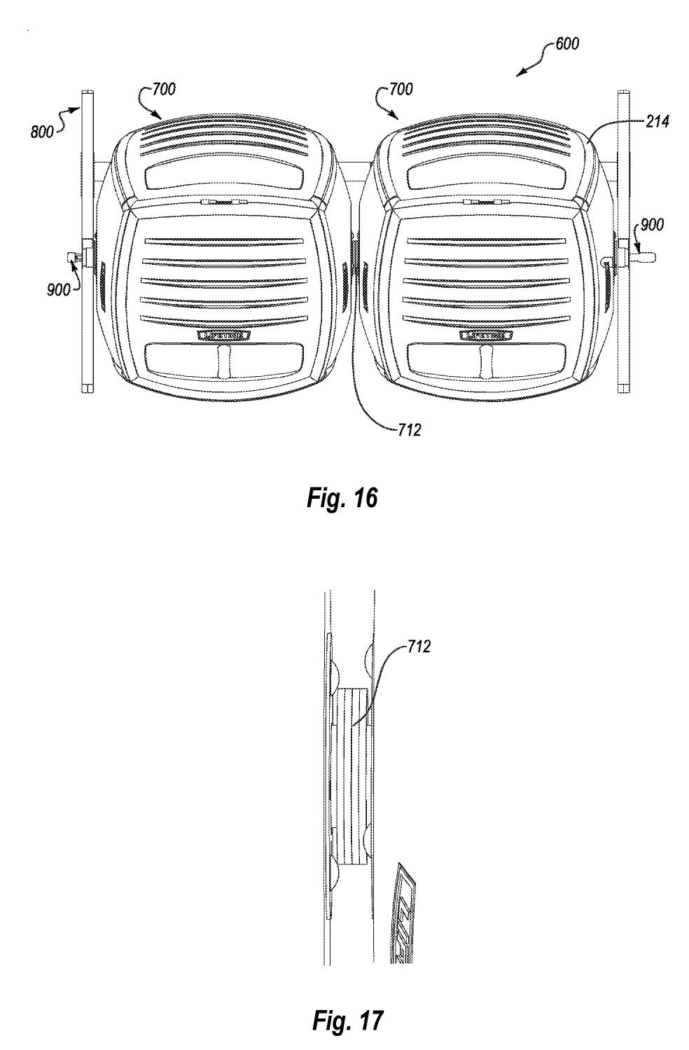

Directing attention, finally, to FIGS. 16 and 17, at least some embodiments of a multi-barrel composter may include one or more spacers 712 positioned between adjacent barrels 700 so as to keep the barrels 700 a desired distance apart from each other. The spacers 712 may be metal or any other suitable material(s), one example of which is polytetrafluoroethylene (PTFE), and may be configured such that the shaft 702 and aeration bar 704 also pass through the spacers 712. The spacers 712 may also provide a relatively low-friction surface that does not materially impair rotation of the barrels 700.

Finally, at least embodiments of the invention that include multiple barrels may include an indicator that a user can use to distinguish the contents of one barrel from the contents of another barrel. For example, a clip may be provided that can attach to the handle or other portion of a barrel. The clip may be colored, such as red for example, to indicate that the contents of the barrel to which the clip is attached are still in the composting process. Another clip, which may be green in color, may indicate that fully composted material is contained in the barrel to which the clip is attached. Any other devices, indicators and/or color schemes may alternatively be employed however.

Although this disclosure has been described in terms of certain embodiments, other embodiments apparent to those of ordinary skill in the art are also within the scope of this disclosure. Accordingly, the scope of the disclosure is intended to be defined only by the claims which follow.

What is claimed is:

1. A composter barrel, comprising:
a first panel having a first complementary structure;
a second panel having a second complementary structure; and
a connector element configured to connect the first and second panels together without the use of fasteners by engaging the first and second complementary structures.

2. The composter barrel as recited in claim 1, wherein the first panel comprises a side panel, and the second panel comprises an end panel having an opening configured to receive a shaft.

3. The composter barrel as recited in claim 1, wherein the first panel and the second panel each comprise a side panel configured to be connected to another side panel and an end panel.

4. The composter barrel as recited in claim 1, wherein each of the panels includes an additional complementary structure configured to engage a connector element.

5. The composter barrel as recited in claim 1, wherein the connector element is configured to releasably engage the first and second complementary structures of the first and second panels.

6. The composter barrel as recited in claim 1, further comprising four additional panels configured to be connected with one or more of the first and second panels by additional connector elements, wherein the six panels are configured to be connected together to form a generally cube-shaped barrel.

7. The composter barrel as recited in claim 1, wherein one of the panels includes interior and exterior surfaces spaced apart from each other and at least partly defining a substantially hollow interior portion.

8. A composter, comprising:
a frame;
a compost barrel, comprising:
a first panel having a first complementary structure;
a second panel having a second complementary structure; and
a connector element configured to connect the first and second panels together without the use of fasteners by engaging the first and second complementary structures; and
a shaft passing through the compost barrel and supported by the frame; and
an adjustment mechanism operable to releasably lock the compost barrel in a desired rotational position relative to an axis defined by the shaft.

9. The composter as recited in claim 8, further comprising an additional compost barrel configured and arranged such that the shaft passes through the additional compost barrel.

10. The composter as recited in claim 8, further comprising:
additional connector elements; and
four additional panels, one or more of which are configured to be connected with one or more other panels by one or more connector elements.

11. The composter as recited in claim 10, wherein the connector elements comprise eight side connector elements, and four corner connector elements, each of the side connector elements configured to connect two panels, and each of the corner connector elements configured to fasten three side connector elements.

12. The composter as recited in claim 10, wherein one of the panels comprises a lid movably attached to one of the remaining five panels, and the five panels are configured to be connected together with the connector elements, the five panels and the lid cooperatively defining a generally cube-shaped compost barrel.

13. The composter as recited in claim 10, wherein the shaft defines a plurality of openings in communication with an interior of the compost barrel.

14. The composter as recited in claim 10, wherein the compost barrel includes a plurality of vents in communication with an interior of the compost barrel.

15. The composter as recited in claim 10, wherein one of the panels comprises a blow-molded element having interior and exterior surfaces spaced apart from each other and at least partly defining a substantially hollow interior portion, the blow-molded element including one or more tack-offs.

16. The composter as recited in claim 10, wherein two of the panels comprise end panels through which the shaft passes, and four of the panels comprise side panels, one of the side panels comprising a lid movably connected to another of the side panels.

17. The composter as recited in claim 16, wherein each of the panels except the lid comprise respective complementary structures configured to engage one or more of the connector elements.

18. The composter as recited in claim 16, further comprising a handle connected to a handle mechanism, the handle mechanism configured and arranged to enable locking and unlocking of the lid by either a clockwise or counterclockwise rotation of the handle.

19. A composter that is at least partly unassembled, the composter comprising the following parts:
a frame;
a shaft configured to be supported by the frame;
a barrel comprising:
first and second end panels each defining an opening configured to receive the shaft;
first, second, and third side panels;
a lid configured to be movably connected to one of the side panels;
eight side connector elements, each of the side connector elements configured to connect two panels;
four corner connector elements, each of the corner connector elements configured to fasten three side connector elements;
a handle connected to a handle mechanism, the handle mechanism configured and arranged to enable locking and unlocking of the lid by either a clockwise or counterclockwise rotation of the handle; and
an adjustment mechanism operable to releasably lock the compost barrel in a desired rotational position relative to an axis defined by the shaft.

20. The composter as recited in claim 19, further comprising an additional barrel.
